(19) United States
(12) Patent Application Publication
(10) Pub. No.: US 2008/0287940 A1
(43) Pub. Date: Nov. 20, 2008

(54) Fiber Pole Tip

(76) Inventor: Lowell D. Hunter, Los Gatos, CA (US)

Correspondence Address: AMS RESEARCH CORPORATION, 10700 BREN ROAD WEST, MINNETONKA, MN, 55343, US

(21) Appl. No.: 12/120,470

(22) Filed: May 14, 2008

Related U.S. Application Data
(60) Provisional application No. 60/917,823, filed on May 14, 2007.

(51) Int. Cl. A61B 18/22

(52) U.S. Cl. 606/16

(57) ABSTRACT

A medical laser unit having a fiber pole tip providing protection and maneuverability to an optical fiber. The fiber pole tip includes a longitudinal channel for retaining the optical fiber so as to limit a bending radius of the optical fiber and prevent the creation of sharp stress risers in the optical fiber. A top surface of the fiber pole tip can include at least one tab projecting over the longitudinal channel to retain the optical fiber within the longitudinal channel. The fiber pole tip is rotatably attached to a fiber pole connected to the laser unit allowing for ease in positioning and manipulating the optical fiber without inducing sharp bends in the optical fiber and to remove stress from the optical fiber.

### PRIORITY CLAIM

The present application claims priority to U.S. Provisional Application Ser. No. 60/917,823 filed May 14, 2007, and entitled, “FIBER POLE TIP”, which is herein incorporated by reference in its entirety.

### FIELD OF THE DISCLOSURE

The invention relates generally to the field of medical laser systems and optical fibers used for the treatment of soft tissue. More specifically, the present invention is directed to a fiber pole tip for a laser system unit that facilitates movement of the optical fiber while simultaneously protecting the optical fiber from damage.

### BACKGROUND OF THE INVENTION

Medical lasers have been used in treatment procedures involving various practice areas including, for example, urology, neurology, otorhinolaryngology, general anesthetic ophthalmology, dentistry, gastroenterology, cardiology, gynecology, and thoracic and orthopedic procedures. Generally, these procedures require precisely controlled delivery laser energy, and often the area to which the energy is to be delivered is located deep within the body, for example, at the prostate or at the fallopian tubes. Due to the location of the target tissue deep within the body, the medical procedure generally requires use of a flexible and maneuverable optical fiber. Depending upon the requirements for a light source, a variety of light sources can be used in conjunction with the optical fiber including, for example, pulsed lasers, diode lasers, and neodymium lasers. Representative lasers used in medical treatment procedures include Ho:YAG lasers and Nd:YAG lasers.

Generally, a surgical probe is utilized in the treatment of body tissue with laser energy. The surgical probe generally comprises an optical fiber coupled to a laser source, wherein the probe is positioned so that the tip of the probe can be positioned adjacent the targeted tissue. Laser energy is directed out of the tip of the optical fiber onto desired portions of the targeted tissue. The laser optical fiber coupled to the laser source is required to be flexible such that the optical fiber can be manipulated to the targeted tissue by a medical professional. However, the flexibility of the optical fiber can contribute somewhat to the possibility of damage to the optical fiber, should it get bumped or crushed.

The laser source or laser unit can be used in a surgical environment, such as in an operating room, or in other environments, such as a clinic or office where out-patient procedures can be performed. The laser unit can comprise a mobile unit capable of being moved from place to place in a surgical or office environment. The optical fiber that extends from the laser unit can be damaged and require replacement when, for example, the optical fiber is being routed from the laser to the patient. Damage to the optical fiber can increase the cost of performing procedures due to the need to replace the optical fiber and potential delay in doing so. Hence, there remains a need for the optical fiber to be protected from damage that can result as the optical fiber is used and routed from the laser unit to the patient.

### SUMMARY OF THE INVENTION

The present invention comprises a fiber pole and fiber pole tip for a laser unit wherein the fiber pole tip retains the optical fiber in the fiber pole tip and provides support to the optical fiber as the optical fiber is being extended between the laser unit and the patient. Use of the fiber pole tip provides support and mobility of the optical fiber without straining or sharply bending the optical fiber.

In one aspect of the present invention, a fiber pole tip is provided wherein the fiber pole tip is largely disc-shaped with a substantially circular top surface. A longitudinal channel traverses the top surface of the fiber pole tip, wherein the channel substantially defines a diameter of the circular top surface of the fiber pole tip. One or more opposed tabs extend from the top surface and over the longitudinal channel to retain the optical fiber in place without straining or placing tension on the optical fiber. Preferably, the fiber pole tip includes at least two opposed tabs having a round configuration to prevent potential damage to the optical fiber. The at least two opposed tabs are preferably offset to retain the optical fiber substantially along a length of the longitudinal channel. By supporting the optical fiber along the length of the longitudinal channel, a potential bending radius of the optical fiber is reduced. In a preferred embodiment, the fiber pole tip rotates on the top of a fiber pole connected to the laser unit. The rotation of the entire fiber pole tip allows for easy positioning of the longitudinal channel/optical fiber during laser treatment and can reduce stress, tension and severe bending that can result from pulling/tugging during a procedure. The fiber pole tip rotates horizontally about an axis defined by the fiber pole

In another aspect of the present invention, the fiber pole tip provides a method of protecting the optical fiber from damage during use. In a first representative step, a laser unit can be provided in which a fiber pole tip having a longitudinal channel is rotatably attached to the laser unit. In a second step, an optical fiber can be connected to the laser unit at a laser output port. In a third step, the optical fiber can be positioned and retained within the longitudinal channel on the fiber pole tip. In a fourth step, the optical fiber can be extended for delivery treatment to a patient whereby the optical fiber slides longitudinally within the longitudinal channel. In a fifth step, the fiber pole tip, and more specifically, the longitudinal channel can be rotatably positioned to remove stress/tension on the optical fiber resulting from handling of the optical fiber during a treatment procedure.

Although specific examples have been illustrated and described herein, it will be appreciated by those of ordinary skill in the art that any arrangement calculated to achieve the same purpose could be substituted for the specific examples shown. For example, other configurations could be substituted for the example handle noted above.

### BRIEF DESCRIPTION OF THE DRAWINGS

These as well as other objects and advantages of this invention will be more completely understood and appreciated by referring to the following more detailed description of the presently preferred exemplary embodiments of the invention in conjunction with the accompanying drawings, of which:

FIG. 1 is a schematic of a representative laser system with a fiber optic attached to the laser unit.

FIG. 2 is a perspective, top view of a laser unit having a fiber pole tip according to an embodiment of the present invention.

FIG. 3 is a perspective view of a prior art pig tail type fiber pole tip attached to a representative laser unit.

FIG. 4 depicts a flow chart illustrating a method of protecting an optical fiber with a fiber pole tip according to an embodiment of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives.

### DETAILED DESCRIPTION OF EMBODIMENTS OF THE INVENTION

The present invention includes a laser unit having a fiber pole tip having a longitudinal channel for receiving an optical fiber. The longitudinal channel generally includes at least two tab members on opposed sides of the longitudinal channel for retaining an optical fiber in the longitudinal channel without placing strain on the optical fiber. The fiber pole tip can be rotatably mounted on a fiber pole such that during maneuvering of the optical fiber, any stress that would typically be placed on the optical fiber is further reduced. In one preferred embodiment, the fiber pole tip is attached to a Greenlight HPS system manufactured by American Medical Systems of Minnetonka, Minn. and as described in U.S. Pat. Nos. 6,554,824 and 6,986,764, which are herein incorporated by reference.

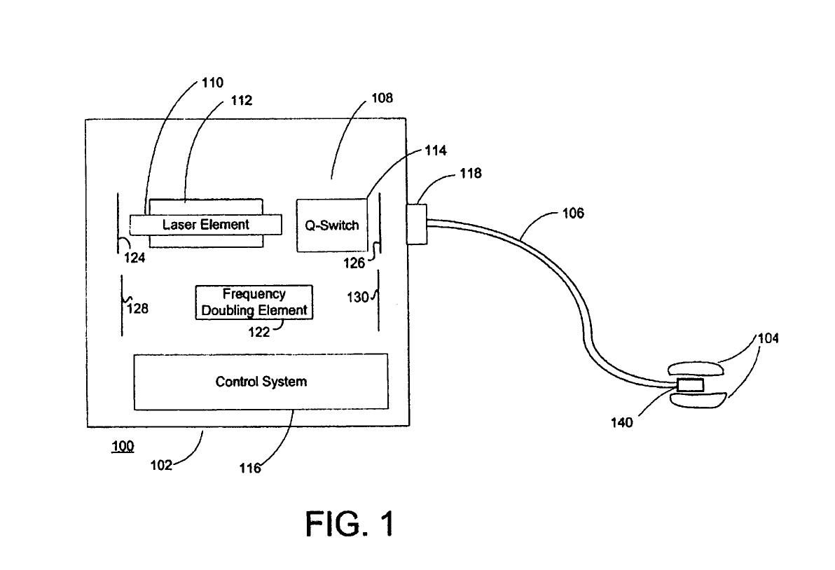

Referring to FIG. 1, there is depicted a block diagram showing an exemplary laser system 100 which may be employed for implementing the present invention. Laser system 100 includes a solid-state laser 102, which is used to generate laser light for delivery through optical fiber 106 to target tissue 104. Laser 102 is capable of being operated in a pulsed mode or continuous wave.

Laser 102 more specifically comprises a laser element assembly 110, pump source 112, and frequency doubling crystal 122. In the preferred-embodiment, laser element 110 outputs 1064 nm light which is focused into frequency doubling crystal 122 to create 532 nm light. According to one implementation, laser element assembly 110 may be neodymium doped YAG (Nd:YAG)crystal, which emits light having a wavelength of 1064 nm (infrared light) when excited by pump source 112. Laser element 110 may alternatively be fabricated from any suitable material wherein transition and lanthanide metal ions are disposed within a crystalline host (such as YAG, Lithium Yttrium Fluoride, Sapphire, Alexandrite, Spinel, Yttrium Orthoaluminate, Potassium Gadolinium Tungstate, Yttrium Orthovandate, or Lanthahum Scandium Borate). Laser element 110 is positioned proximal to pump source 112 and may be arranged in parallel relation therewith, although other geometries and configurations may be employed.

Pump source 112 may be any device or apparatus operable to excite laser element assembly 110. Non-limiting examples of devices which may be used as pump source 112, include: arc lamps, flashlamps, and laser diodes.

A Q-switch 114 disposed within laser 102 may be operated in a repetitive mode to cause a train of micropulses to be generated by laser 102. Typically the micropulses are less than 1 microsecond in duration separated by about 40 microseconds, creating a quasi-continuous wave train. Q-switch 114 is preferably of the acousto-optic type, but may alternatively comprise a mechanical device such as a rotating prism or aperture, an electro-optical device, or a saturable absorber.

Laser 102 is provided with a control system 116 for controlling and operating laser 102. Control system 116 will typically include a control processor which receives input from user controls (including but not limited to a beam on/off control, a beam power control, and a pulse duration control) and processes the input to accordingly generate output signals for adjusting characteristics of the output beam to match the user inputted values or conditions. With respect to pulse duration adjustment, control system 116 applies an output signal to a power supply (not shown) driving pump source 112 which modulates the energy supplied thereto, in turn controlling the pulse duration of the output beam.

Although FIG. 1 shows an internal frequency doubled laser, it is only by way of example. The infrared light can be internally or externally frequency doubled using non-linear crystals such as KTP, Lithium Triborate (LBO), or Beta Barium Borate (BBO) to produce 532 nm light. The frequency doubled, shorter wavelength light is better absorbed by the hemoglobin and char tissue, and promotes more efficient tissue ablation. Finally, the green light leaves only a thin char layer with little pre and post operative bleeding.

Laser 102 further includes an output port 118 couplable to optical fiber 106. Output port 118 directs the light generated by laser 102 into optical fiber 106 for delivery to tissue 104. Mirrors 124, 126, 128, and 130 direct light from the lasing element 110 to the frequency doubling crystal 122, in addition to forming the resonant cavity of the laser. Mirrors 124, 126, 128, and 130 are configured for focusing the light to form an image just in front of the frequency doubling crystal 122 on the side closer to mirror 130, and to compensate for thermal lensing in the lasing element. Although mirrors 124, 126, 128, and 130 are illustrated as flat and parallel to the walls of the laser, typically the focusing is achieved by curving and/or angling the mirrors. Alternatively transmissive optical elements could be used to focus the light and compensate for the thermal imaging. Mirrors 124, 128 and 130 reflect both the wavelength of light produced by the lasing element (e.g. 1064 nm) and the wavelength of the frequency doubled light (e.g. 532 nm). Mirror 126 only reflects the light originating from the lasing element 110 (e.g. 1064 nm) but is transparent to the frequency doubled light (e.g. 532 nm), forming an output window. Higher harmonic outputs may also be generated from the 1064 nm line, or other line amplified in the laser, including third and fourth harmonics, for shorter wavelengths. Other laser systems may be used, including but not limited to Sapphire lasers, diode lasers, and dye lasers, which are adapted to provide the output power and wavelengths described herein, including wavelengths in the ranges from 200 nm to 1000 nm and from 1100 nm to 1800 nm, for example.

While a bare fiber may be utilized for certain procedures, optical fiber 106 preferably terminates in a tip 140 having optical elements for shaping and/or orienting the beam emitted by optical fiber 106 so as to optimize the tissue ablation process. In the instance of treating BPH, the tip is preferably a side-firing tip. At times it is necessary to physically move the laser unit 100 between different treatment locations. In addition, it is often necessary to move or otherwise reposition the optical fiber 106 for best access to the patient and the target tissue 104. In moving the laser unit 102, it is important to avoid bending or kinking the optical fiber 106 such that signal transmission is lost. Further, it is also important not to bend or induce stress in the optical fiber 106 when manipulating and positioning the optical fiber 106 for use, otherwise the optical fiber 106 can become damaged and unusable.

At times of use, the optical fiber 106 is disposed in an extended position, stretching from the laser unit 102 to the patient. The optical fiber 106 can then be manipulated into position to accomplish a required task at the target tissue 104. The task may include, for example, insertion through a bodily incision or orifice to ablate particular tissue. The optical fiber 106 may need to be positioned and repositioned a number of times during a medical laser procedure.

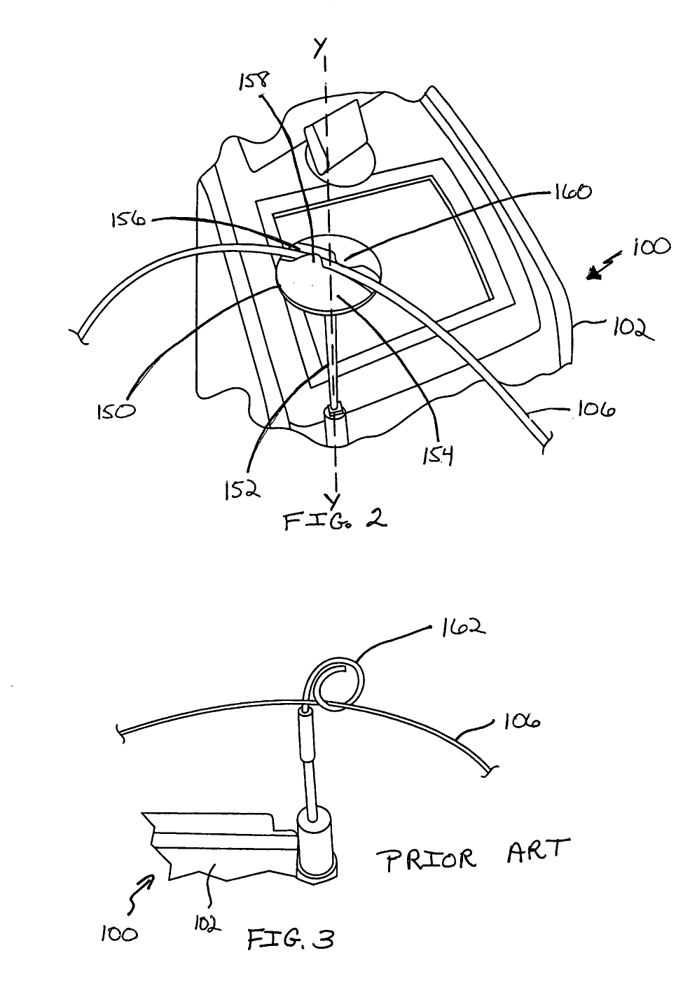

Referring now to FIG. 2, a fiber pole tip 150 is affixed to a distal end of a fiber pole 152 and is constructed and positioned on the laser unit 102 to facilitate manipulation of the optical fiber 106 to prevent straining or undue bending of the optical fiber 106. The fiber pole tip 150 is generally disk-shaped, with a substantially circular top surface 154. A longitudinal channel 156 traverses the circular top surface 154 of the fiber pole tip 150. The longitudinal channel 156 substantially forms the diameter of the top surface 154 of the fiber pole tip 150. The longitudinal channel 156 is preferably at least two inches long so as to support a substantial portion of the optical fiber 106, thereby limiting a bending radius of the optical fiber 106 so as to prevent sharp stress risers. The top surface 154 of the fiber pole tip 150 includes at least two opposed tabs 158, 160 with at least one on each side of the longitudinal channel 156. Preferably, the at least two opposed tabs 158, 160 are rounded so as to avoid edges that can damage the optical fiber 106. The opposed tabs 158, 160 are extensions of the top surface 154 and at least partially extend over the longitudinal channel 156. Further, the opposed tabs 158, 160 are offset from one another across the longitudinal channel 156. The longitudinal channel 156 has a sufficient depth to accommodate the thickness of the optical fiber 106 without constraining longitudinal, slidable movement of the optical fiber 106 through the longitudinal channel 156. The optical fiber 106 slides longitudinally along the length of the longitudinal channel 156, while the opposed tabs 158, 160 both guide and contain the optical fiber 106 within the longitudinal channel 156. The optical fiber 106 slidably moves through the longitudinal channel 156, as the optical fiber 106 is extended during use, without leaving the confines of the longitudinal channel 156 and the fiber pole tip 150. The support provided to the optical fiber 106 by the fiber pole tip 150 reduces the amount of stress to which the optical fiber 106 may be subjected, especially as compared to a conventional, prior art “pig tail” fiber pole tip 162 as shown in FIG. 3. To further reduce stress placed on the optical fiber 106, the entire fiber pole tip 150 is horizontally rotatable about an axis y-y defined by the fiber pole 152. The rotation of the fiber pole tip 150 allows repeated positioning and repositioning of the direction of the fiber pole tip 150 and, hence, the longitudinal direction of the optical fiber 106.

In operation, the proximate end of the optical fiber 106 is connected to the laser unit 102 through the output port 118. A distal end of the optical fiber 106 is guided through the longitudinal channel 156, traversing the top surface 154 of the fiber pole tip 150. Upon exiting the longitudinal channel 156, the optical fiber 106 is extended to the patient. Alternatively, the optical fiber 106 can be positioned on top surface 154 of the fiber pole tip 150 and the optical fiber 106 is pressed past the two opposed tabs 158, 160 into the longitudinal channel 156. In another alternative method, the optical fiber 106 can be positioned in the longitudinal channel 156 of the fiber pole tip 150 by stringing the optical fiber 106 through the longitudinal channel 156 or by pressing the optical fiber 106 past the opposed tabs 158, 160 and then connecting the optical fiber 106 to the laser unit 102. Once connected, the optical fiber 106 is able to be slidably extended or rotatably positioned with minimal bending of the optical fiber 106 due to the combination of the configuration of the fiber pole tip 150 on the fiber pole 152.

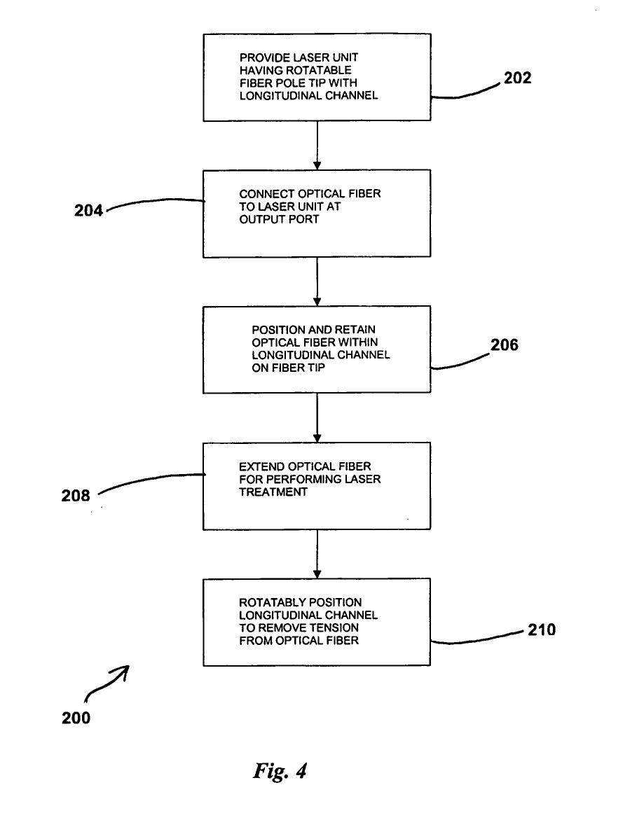

A representative method 200 of the present invention is illustrated schematically in FIG. 4. In a first step 202, the laser unit 102 can be provided with a fiber pole tip 150 rotatably attached to a fiber pole 152 on the laser unit 102. In a second step 204, the optical fiber 106 can be connected to the laser unit 102 at the output port 118. In a third step 206, the optical fiber 106 can be positioned within the longitudinal channel 156 such that the opposed tabs 158, 160 retain the optical fiber 106. Positioning the optical fiber 106 within the longitudinal channel 156 can involve sliding the optical fiber through one end of the longitudinal channel 156 and out and an opposed end or by pressing the optical fiber 106 past the opposed tabs 158, 160 and into the longitudinal channel 156. In a fourth step 208, the optical fiber 106 can be longitudinally extended through the longitudinal channel 156 such that the optical fiber 106 is in position for accomplishing a laser treatment. In a fifth step 210, the fiber pole tip 150 can be rotatably positioned about the fiber pole 152 to remove any tension in the optical fiber 106 during positioning of the optical fiber 106.

Although specific examples have been illustrated and described herein, it will be appreciated by those of ordinary skill in the art that any arrangement calculated to achieve the same purpose could be substituted for the specific examples shown. This application is intended to cover adaptations or variations of the present subject matter. Therefore, it is intended that the invention be defined by the attached claims and their legal equivalents.

We claim:

1. A medical laser system comprising:
a laser unit having an optical fiber, the laser unit including a fiber pole having a fiber pole tip rotatably attached to the fiber pole, the fiber pole tip including a longitudinal channel defined therein such that the optical fiber can be retainably positioned within the longitudinal channel.

2. The medical laser system of claim 1, wherein the optical fiber is slidably movable through the longitudinal channel.

3. The medical laser system of claim 1, wherein the longitudinal channel is rotatably positioned to relieve stress on the optical fiber.

4. The medical laser system of claim 1, wherein the longitudinal channel has a channel depth greater than the optical fiber diameter such that the optical fiber is fully contained therein.

5. The medical laser system of claim 1, wherein a top surface of the fiber pole tip further comprises at least one tab extending at least partially over the longitudinal channel.

6. The medical laser system of claim 5, wherein at least one tab comprises at least two tabs, wherein at least one tab is positioned on a first side of the longitudinal channel and at least one tab is positioned on a second side of the longitudinal channel.

7. The medical laser system of claim 6, wherein the at least two tabs retain the optical fiber in the channel.

8. The medical laser system of claim 1, wherein the optical fiber is retained along a length of the longitudinal channel to limit a bend radius of the optical fiber.

9. A method of protecting an optical fiber connected to a laser unit comprising:
providing a laser unit having a fiber pole tip connected thereto;
attaching an optical fiber to the laser unit; and
positioning the optical fiber in a longitudinal channel on the fiber pole tip.

10. The method of claim 9, wherein providing the laser unit having the fiber pole tip connected thereto comprises rotatably mounting the fiber pole tip on a fiber pole connected to the laser unit.

11. The method of claim 10, further comprising:
rotating the fiber pole tip so as to position the longitudinal channel to relieve stress on the optical fiber.

12. The method of claim 9, wherein positioning the optical fiber in the longitudinal channel comprises pressing the optical fiber past a plurality of opposed tabs above the longitudinal channel.

13. The method of claim 9, wherein positioning the optical fiber in the longitudinal channel comprises sliding the optical fiber from a first end of the longitudinal channel to a second end of the longitudinal channel.

14. The method of claim 9, further comprising:
limiting a bend radius of the optical fiber within the longitudinal channel by retaining the optical fiber along a length of the longitudinal channel.
